(19) United States
(12) Patent Application Publication
(10) Pub. No.: US 2014/0276810 A1
(43) Pub. Date: Sep. 18, 2014

(54) DEVICES FOR TISSUE RESECTION

(71) Applicant: BOSTON SCIENTIFIC SCIMED, INC., Maple Grove, MN (US)

(72) Inventors: Samuel RAYBIN, Marlborough, MA (US); Naroun SUON, Lawrence, MA (US); Kenichi ADACHI, Sagamihara-shi (JP); Paul SMITH, Smithfield, RI (US)

(73) Assignee: BOSTON SCIENTIFIC SCIMED, INC., Maple Grove, MN (US)

(21) Appl. No.: 14/209,835

(22) Filed: Mar. 13, 2014

Related U.S. Application Data
(60) Provisional application No. 61/785,214, filed on Mar. 14, 2013.

(51) Int. Cl. A61B 18/14

(52) U.S. Cl. CPC A61B 18/14
USPC 606/46

(57) ABSTRACT

A device for tissue resection and methods of using the same is disclosed, including a snare loop and a tubular handle. The snare loop has both conductive and insulated portions such that the snare loop can be used to cut tissue selectively to make perimeter cuts on the tissue to be resected. The snare loop may be placed and tightened on a target tissue and may be activated for cutting the tissue to be resected by passing an electrical current through the snare loop. Some embodiments of the device include a hood and cutting wire for making perimeter cuts on the tissue to be resected.

### CROSS-REFERENCE TO RELATED APPLICATIONS

This application claims the benefit of priority from U.S. Provisional Application No. 61/785,214, filed on Mar. 14, 2013, the entirety of which is incorporated by reference herein.

### FIELD OF THE DISCLOSURE

Embodiments of the present disclosure relate generally to medical devices and procedures. Particularly, embodiments of the present disclosure relate to tissue resection through minimally invasive techniques.

### BACKGROUND OF THE INVENTION

Tissue resection procedures, such as colonoscopy and polypectomy, are carried out by inserting introduction instruments, such as endoscopes or laparoscopes, into the body of a patient through incisions or natural anatomical openings. Commonly, such devices employ snares, typically designed as loops, for performing tissue resection procedures.

For example, piecemeal endoscopic mucosal resection (EMR) is a state-of-the-art endoscopic method used to remove large benign lesions from the human gastrointestinal tract. In piecemeal EMR, small pieces of lesion (˜10 mm) are removed by snares until the lesion is completely resected. In contrast, en bloc resection is resection of an entire large bulky tumor or infected portion without dissection.

En bloc resection is sometimes preferred because the histological analysis of the resected specimen can be performed thoroughly. By contrast, piecemeal EMR may be preferred to en bloc resection techniques, such as endoscopic submucosal dissection (ESD) because it requires significantly less skill and training. However, since small portions of lesion can remain unresected, piecemeal resection has a higher recurrence rate than en bloc removal of the lesion.

The present disclosure has various advantages over known tissue resection techniques.

### SUMMARY OF THE INVENTION

The invention generally relates to devices and methods for making perimeter cuts around a tissue for resection.

Embodiments of the present disclosure relate to medical systems for performing tissue resection. In one aspect, a device is disclosed with a snare loop having a distal end and a tubular handle movably coupled to the snare loop away from the distal end. The snare loop has one or more insulated portions and one or more conductive portions, and can be activated by passing an electrical current through the snare loop. The snare loop a) can be drawn around a tissue to be resected, b) can be activated to cut the tissue in contact with the conductive potions of the snare loop, and c) can recede into and advance from a cavity in the tubular handle. In some embodiments, the snare loop has an insulated portion located toward the distal end of the snare loop. In some embodiments, the device includes an instrument to raise the target tissue such as an injected bleb, a suction cap, or a band ligator.

In one aspect, the snare loop has a conductive portion located at the distal end which can be used to cut the tissue to be resected in contact with the conductive portion when the snare loop is activated. The insulated portions of the snare loop may include a fork shaft and two prongs. In some embodiments, the insulated portions of the snare loop have one or more inner lumens and the conducting portion can recede into and advance from the lumens. The insulated portions of the snare loop may be integrated with the handle, wherein the lumens of the insulated portions are coupled to the cavity of the handle and the conducting portion can recede into and advance from the lumens and the cavity.

In some embodiments, the snare loop is configured to transition between at least one extended state and at least one retracted state such that the length of the conductive portion is enlarged in the extended state, and the length of the conductive portion is reduced in the retracted state.

In one aspect, a resection device is disclosed having a hood and a cutting wire. The hood has a cavity, an inner surface and two or more holes on the inner surface such that the hood is capable of capturing a target tissue by applying vacuum or pressure. The cutting wire extends between the holes of the hood in such a way that the cutting wire a) forms one or more resecting units along the inner surface or within the cavity of the hood, and b) the cutting wire is activated to cut tissue to be resected in contact with the wire. The cutting wire may be conductive and may be activated by passing an electrical current through the wire. In some embodiments, the cutting wire makes curved perimeter cuts on the tissue to be resected. The hood may be movably coupled to an introduction sheath such that the position of the cutting wire can be extended distally and be contracted proximally along the sheath to facilitate capturing of tissue within the hood and/or cutting the tissue for resection by the cutting wire. In some embodiments, the resection device may be attached to one end of an endoscope. The cutting wire may be an actuation element or an electrical path for cautery cutting.

In some embodiments, the cutting wire may be configured to move between at least one extended state and at least one retracted state. The cutting wire may lie along the inner surface of the hood in the extended state and may be positioned to bisect the cavity of the hood in the retracted state. The hood may have a groove along the inner surface of the hood where the cutting wire can be lodged in the extended state.

In one aspect, a method is disclosed with the following steps: (a) placing a snare loop around a target tissue via a tubular handle having an inner cavity, the snare loop having one or more insulated portions and one or more conductive portions; (b) tightening the snare loop around the target tissue by retracting a part of the snare loop inside the cavity of the handle; and (c) activating the snare loop by passing electric current to cut tissue to be resected in contact with the conductive portions of the snare loop to make at least a partial perimeter cut around the tissue to be resected. The method may include making a plurality of perimeter cuts around the tissue to be resected such that the resection of the tissue is facilitated. Subsequently, the tissue to be resected may be removed by en bloc techniques after making a plurality of perimeter cuts. In addition, an injected bleb, a suction cap, or a band ligator may be used to raise the target tissue.

Additional objects and advantages of the present disclosure will be set forth in part in the description which follows, and in part will be understood from the description, or may be learned by practice of the claimed invention. The objects and advantages of the claimed invention will be realized and attained by means of the elements and combinations particularly pointed out in the appended claims.

It can be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1A is a schematic view of an exemplary device in an extended state, according to an embodiment of the present disclosure.

FIG. 1B is a schematic view of an exemplary device in a retracted state, according to an embodiment of the present disclosure.

FIG. 1C is a schematic view of an exemplary device placed around a target tissue, according to an embodiment of the present disclosure.

FIG. 1D is a schematic view of an exemplary device tightened around the target tissue, according to an embodiment of the present disclosure.

FIG. 1E is a schematic view of an exemplary device activated with electrical current to cut the tissue to be resected, according to an embodiment of the present disclosure.

FIG. 1F is a schematic view of a curved incision made on tissue to be resected, according to an embodiment of the present disclosure.

FIG. 1G is a schematic view showing a plurality of curved perimeter cuts made around a lesion, according to an embodiment of the present disclosure.

FIG. 2A is a schematic view of an exemplary device in an extended state, according to an embodiment of the present disclosure.

FIG. 2B is a schematic view of an exemplary device in a retracted state, according to an embodiment of the present disclosure.

FIG. 2C is a schematic view of an exemplary device in another retracted state, according to an embodiment of the present disclosure.

FIG. 3A is a schematic view of an exemplary resection device with a cutting wire in a first state, according to an embodiment of the present disclosure.

FIG. 3B is a schematic view of an exemplary resection device with the cutting wire in a second state, according to an embodiment of the present disclosure.

### DESCRIPTION OF THE EMBODIMENTS

Reference will now be made in detail to embodiments of the present disclosure, examples of which are illustrated in the accompanying drawings. Wherever possible, the same reference numbers will be used throughout the drawings to refer to the same or like parts. The terms “distal”, “distally” or “distal end” may refer to the direction or end farthest away from a user when introducing a device into a patient. Thus, the distal direction is relatively closer to the body or infected portion of the patient as compared to the proximal direction that is closer to the operator or user. The term “cavity” may refer to, for example, a hollow or a vacant portion within a cylindrical or tubular structure. The term “target tissue” may refer to tissue that is target to make an incision or perimeter cut. The target tissue may be part of tissue that is to be removed, such as a lesion or near or around such tissue. The term “tissue to be resected” may refer to, for example, tissue or part of an organ that is to be removed from the body, such as infected tissue, lesions, tumors, cancerous polyps, infected organs, or parts of, organs, etc. Further, the term “lumen” may refer to, for example, an interior of a tubular structure.

The term “endoscope” may refer to, for example, a device that is used to illuminate an organ or object under inspection, in which a light source is normally outside the body and light can be directed through an optical fiber system. The endoscope may have a distal end towards or inside the body of a patient, a proximal end away from the body and handled by a controller or user, and a lumen extending between the ends. Further, the endoscope may have an elongable flexible insertion section and the controller may be able to manipulate the distal end of the endoscope.

The term “fork shaft” may refer to, for example, the base of a fork-like structure to which two or more protrusions or prongs are attached. The term “prongs” may refer to, for example, the protrusions of the “fork shaft.” The term “extended state” may refer to, for example, one or more positions where the snare loop or conductive portion is in a loosened or elongated, such as into an expanded state. In this state, the snare loop or resection device can be placed on a target tissue. The term “retracted state” may refer to, for example, a state in which the snare loop or conductive portion is tightened or reduced in circumference or length, such as into a collapsed state. The term “hood” may refer to, for example, a body (e.g., cylindrical) that may be used to at least partially surround (e.g., encircle) a target tissue, and in which a vacuum or mechanical pressure can be applied on a target tissue. The hood may be attached to, or a part of, an endoscope. The term “introduction sheath” may refer to, for example, an encasing structure that may be attached to one end of an endoscope, which is inserted into the body. The term “mucosa” may refer to, for example, a surface layer of an organ wall. The term “muscularis” may refer to, for example, a muscle layer inside a human body. The term “cautery” may refer to, for example, burning a part of body to remove or destroy a tissue. The term “cautery cutting” may refer to, for example, a surgical procedure for producing a smooth cut with minimum tissue damage.

### Overview

Embodiments of the present disclosure relate to medical devices for performing resection, including removing or retrieving tissue by making small perimeter cuts around the tissue to be resected. For example, embodiments of the disclosed device may facilitate removal of unwanted tissue, such as cancerous polyps or lesions, from within a patient's body, including tissue disposed on, e.g., the mucosal walls of the colon, esophagus, stomach, or duodenum. A physician may also desire to resect tissue in order to conduct a biopsy or other examination. It should be noted that the present devices can be used both for retrieving and for severing target tissue or objects.

In one embodiment, a device for tissue resection may generally include a snare loop and a tubular handle. The snare loop may have both conductive and insulated portions, such that the snare loop can be used to cut tissue selectively to make perimeter cuts on the tissue to be resected. The snare loop may be placed and tightened on a target tissue and may be activated for cutting the tissue to be resected by passing an electrical current through the snare loop. Some embodiments of the device include a hood and cutting wire for making perimeter cuts on the tissue to be resected.

The conductive portions of the snare loop or the cutting wire may be made from conductive non-toxic metals such as copper, aluminum, etc. In addition, the conductive material may be constructed from other materials with a metal coating or a conductive polymer coating. The snare loop wire may include a braided wire, multiple wires, or other suitable wires known to those skilled in the art. In addition, the material employed to manufacture such wires may include, but not be limited to, a rigid, a flexible, or a semi-rigid material. Exemplary materials may include metals, polymers, composites, alloys, or the like. In one embodiment, the snare loop wire may be made of a suitable biocompatible material, e.g., stainless steel or nitinol. In contrast, the insulated portions of the snare loop may include an insulated rubber, composite materials or polymer sleeve. In one embodiment, the insulated portions may be coated with a suitable friction reducing material such as TEFLON®, polyetheretherketone, polyimide, nylon, polyethylene, or other lubricious polymer coatings, to reduce surface friction with the surrounding tissues. The insulating portions may include a hydrophilic layer of polymers known in the art, to prevent inadvertent cauterizing of surrounding tissue. Further, insulated and/or noninsulated portions of the snare loop wire may be coated with an antimicrobial covering to inhibit any microbial growth on its surface. For instance, the coating may include an anti-bacterial covering, which may contain an inorganic antibiotic agent, disposed in a polymeric matrix that adheres the antibiotic agent onto the surface of the snare loop. The handle and other parts of the device may be constructed from any known materials such as a non-toxic metal, polymer, ceramic materials.

Various embodiments of a device for resection of tissue are disclosed in the following examples.

### Exemplary Embodiments

FIGS. 1A-1G depict an exemplary device 100 having a shaft 106 having a proximal end 105 and a snare loop 107 at a distal end. As shown in FIG. 1A, the snare loop 107 may have an insulated portion 104 near or around a distal end 103, and a conductive portion 102 located between the insulated portion 104 and the shaft 106. In other words, the snare loop 107 may have conductive portions 102 on both sides of the loop, and on either end of insulated portion 104. In one embodiment, the device 100 may be configured to move between at least one extended state, in which the snare loop 107 is in an expanded position allowing the snare loop to encircle target tissue, and at least one retracted state, in which the snare loop 107 is collapsed or tightened around the target tissue. FIG. 1A depicts device 100 in an extended state with snare loop 107 in an expanded state, whereas FIG. 1B depicts device 100 in a retracted state with snare loop 107 in a collapsed or tightened state.

As shown in FIG. 1C, the device 100 can be positioned around target tissue, depicted here as target tissue 108. The target tissue may be a part of or around the infected tissue that is to be retrieved or removed. As shown in FIG. 1D, the snare loop 107 may be tightened around the target tissue 108. Thus, both conductive portions 102 and the insulated portion 104 of the snare loop may be urged in contact with the target tissue. Retracting or collapsing snare loop 107 around target tissue 108 may urge the target tissue in better contact with the snare loop, as well as urge the target tissue into and through the plane of the snare loop. Auxiliary instruments, such as an injected bleb, a suction cap, or a band ligator, may be used to urge the target tissue into and through the plane of the snare loop.

In one embodiment, tightening or collapsing of snare loop 107 may be facilitated by retraction of the snare loop wire into shaft 106, or by advancement of the shaft 106 and/or an outer sheath of shaft 106 toward the distal end 103, or by any other known methods. In some embodiments, when snare loop 107 is retracted or otherwise manipulated into a collapsed position, some or all of the conductive portion 102 may be retracted inside a lumen of shaft 106. The snare loop 107 may be retracted partially or fully depending on the size and type of target tissue. Tightening of the snare loop around the target tissue facilitates good contact between the conductive and insulated portions of the snare loop 107 and the tissue, in preparation for cutting the tissue.

As shown in FIG. 1E, the device 100 may be activated such that tissue incision is achieved. In some embodiments, electrical current is passed through the snare loop 107 such that the conductive potions 102 cut through the tissue 108 while the tissue in contact with the insulated portion 104 is left intact. It is envisioned that the tissue incision may be accomplished by various methods in addition to activation by passing electrical current. For example, the incision may be facilitated by using a sharpened edge, such as a cutter, along some or all of conductive portions 102, and applying mechanical pressure on the tissue by tightening the snare loop 107.

Subsequently, when the snare loop 107 is released, a curved incision 110 may be made in the target tissue, as shown in FIG. 1F. The curvature and size of the incision can be controlled based on the size and shape of the snare loop. In one embodiment, curved incisions can be used to make small perimeter cuts around a lesion or infected tissue. As shown in FIG. 1G, perimeter cuts 110, 120, 130, 140 and 150 may be made around a lesion or infected tissue 160. The perimeter cuts may be partial, semi-circular, circular, or any other desired shape. The tissue to be resected 170 (e.g., resected in the direction pointed by the arrow) may be smaller or larger than the area of the lesion or infected tissue 160. Once a perimeter cut (e.g., a curved cut or resection along a perimeter of the lesion) is made, an entire lesion (e.g., infected or diseased portion) can be easily resected in an en bloc sequence using endoscopic submucosal dissection (ESD) or large snare techniques. It is to be noted that FIG. 1G is a schematic and the perimeter cuts may not completely encircle the tissue or infected tissue in practice. In addition, a permiter cut may form any regular or irregular shape around the tissue.

FIGS. 2A-2C depict a device with the insulated portions 204 forming a forked structure with a forked shaft 210 and two insulated prongs 206 and 207. A conductive portion 202 is located at a distal end 203 of the device 200. The insulated portions 204 and the conductive portions 202 together form the snare loop 209. In some embodiments, the insulated portions 204 are integrated with the shaft 205. FIG. 2A depicts the snare loop in an extended state where the conductive portion 202 is fully exposed. In this position, the length of the conductive portion 202 is relatively enlarged and the device can be placed around a target tissue (not shown). Further, as shown in FIG. 2B, when in a retracted state, the snare loop 209 is tightened as the length of the conductive portion 202 is reduced. When the snare loop 209 is tightened, the device can tighten around the target tissue (not shown). In some embodiments, the conductive portion 202 can recede into and advance from one or more inner lumens of the insulated portions 204. In some embodiments, the shaft 205 may be a tubular member having a cavity and the lumens of the insulated portions 204 are coupled with or in communication with the cavity such that the conductive portion 202 can recede into and advance from the lumen and cavity of shaft 205. The tightening of the conductive portion 202 can be achieved by receding the conductive portion within the lumen of the insulated portions 204 and/or cavity of the shaft 205. Practically, this tightening may be achieved in many ways, such as by pulling on a conductive wire that is placed within the lumen of the insulated portions 204 or by advancing the position of the shaft 205 distally, for example. In this way, the length of the conductive portion may be reduced in the retracted state so that the area enclosed by the snare loop 209 is reduced to tighten around the tissue (not shown). Tightening of the snare loop around the target tissue facilitates good contact between the conductive and insulated portions of the snare loop in preparation for cutting the tissue.

After the snare loop 209 is tightened around the target tissue, the snare loop may be activated to make a cut or incision on the tissue by activating the snare loop as described above. For example, activation of the snare loop may be achieved by passing an electrical current through the snare loop such that the tissue in contract with the conductive portion 202 is cut. In one embodiment, tightening of the snare loop around the target and/or passing an electrical current through the snare loop may initiate making permiter cuts in the tissue.

In FIG. 2C, the snare loop 209 is shown in a further retracted state, where only a small length of the conductive portion 202 is exposed. In some embodiments, this tightening is achieved by tightening the length of the conductive portion 202 and bringing the two prongs 206 and 207 together. In this retracted position, the conducting wire may be used to apply mechanical pressure on the tissue to be resected to facilitate cutting of the tissue. The snare loop 209 may still be activated to cut tissue in this position. Using such a device, curved incisions may be made as shown in FIGS. 1F and 1G.

FIGS. 3A and 3B depict a resection device 300 including a hood 309 and a cutting wire 302. The hood may be a suction or cutting hood where a target tissue (not shown) can be encircled within by applying vacuum or pressure. Suctioning the tissue also has the advantage of raising the target tissue and creating good contact between the hood 309 and the target tissue. As shown in FIG. 3A, the cutting wire 302 extends from two holes 304 and 306 some distance apart on the inner surface of the hood 309 forming a resecting unit.

In some embodiments, the hood 309 is attached to or part of an introduction sheath that is attached to an instrument that can be introduced into a body. For example, the hood 309 may be attached to one end of an endoscope so that a user can use such a modified instrument for tissue resection. The position of sheath may be adjustable such that the sheath can be extended distally or towards the distal direction (310) and contracted proximally or in the proximal direction (320). Such positioning of the sheath may facilitate capturing of tissue within the hood 309 and/or cutting of the tissue to be resected.

The cutting wire 302 may be used to cut the tissue to be resected after it is activated. For example, the cutting wire may be conductive and may be activated by passing electrical current through it to cut the tissue to be resected. It is envisioned that the cutting wire 302 may need to be electrically isolated from the rest of the device 300 using an insulated sleeve placed along the holes 304 and 306. The cutting wire 302 can be configured as an actuation element and an electrical path for cautery cutting.

Further, as shown in FIG. 3B, the cutting wire 302 may be in a retracted state as compared to an extended state as depicted in FIG. 3A. In FIG. 3A, the cutting wire 302 follows inner surface of the hood 309 in a curved shape such that it can make curved perimeter cuts on the tissue to be resected. Further, the cutting wire 302 may also lie in a groove 305 along the inner surface of the suction hood 309 as shown in FIG. 3B. Consequently, when the cutting wire 302 is retracted and pulled taught, the wire 302 spans a portion of hood 309 lying between the two holes. In contrast, in the retracted position, the cutting wire 302 is positioned to bisect the cavity or hollow inner surface of the hood 309. In this position, the hood 309 can be used to make relatively straight-line incisions on the tissue to be resected.

Advantages of the embodiments include devices and methods for making a plurality of small perimeter cuts on tissue to be resected in an easy and safe manner. Using such techniques and devices, users are able to remove large lesions after making several small perimeter cuts around a tissue. Embodiments of the present disclosure may be used in any medical or non-medical procedure, including any medical procedure where appropriate resection of undesired body tissue is required. In addition, at least certain aspects of the aforementioned embodiments may be combined with other aspects of the embodiments, or removed, without departing from the scope of the disclosure.

Other embodiments of the present disclosure will be apparent to those skilled in the art from consideration of the specification and practice of the embodiments disclosed herein. It is intended that the specification and examples be considered as exemplary only, with a true scope and spirit of the invention being indicated by the following claims.

What is claimed is:

1. A device comprising:
a snare loop having a distal end and a proximal and, a single insulated portion at one of the distal end and the proximal end, and a single conductive portion at the other of the distal end and the proximal end, wherein the snare loop is configured to be drawn around a target tissue to be resected, and activated with an electrical current to cut tissue in contact with the conductive portions of the snare loop; and
a tubular shaft movably coupled to the snare loop away from the distal end such that the snare loop is configured to recede into and advance out of an inner cavity in the tubular shaft.

2. The device of claim 1, wherein the single insulated portion is at the distal end of the snare loop.

3. The device of claim 1, further comprising an instrument configured to raise the target tissue.

4. The device of claim 1, wherein the single conductive portion is at the distal end and configured to cut tissue in contact with the conductive portion when the snare loop is activated.

5. The device of claim 4, wherein the insulated portion of the snare loop includes a forked shaft and two prongs.

6. The device of claim 5, wherein the forked shaft and two prongs include one or more inner lumens, and the conducting portion is configured to recede into and advance out of the one or more inner lumens.

7. The device of claim 6, wherein the forked shaft and two prongs are integral with the tubular shaft; wherein the one or more inner lumens are provided in communication with the cavity of the shaft; and wherein the conducting portion is configured to recede into and advance out of the cavity.

8. The device of claim 6, wherein the snare loop is configured to transition between at least one extended state and at least one retracted state such that a length of the conductive portion is enlarged in the extended state, and the length of the conductive portion is reduced in the retracted state.

9. A resection device comprising:
a hood having a cavity, an inner surface, and two or more holes on the inner surface, wherein the hood is configured to capture a target tissue by applying vacuum or pressure to the target tissue; and
a cutting wire extending between the two or more holes of the hood, wherein the cutting wire forms a resecting unit along the inner surface or within the cavity of the hood, and the cutting wire is activated to cut tissue in contact with the cutting wire.

10. The resection device of claim 9, wherein the cutting wire is conductive and is activated by passing an electrical current through the wire.

11. The resection device of claim 9, wherein the cutting wire is configured to make curved perimeter cuts in the tissue to be resected.

12. The resection device of claim 9, wherein the hood is movably coupled to an introduction sheath, and the position of the cutting wire can be extended distally and contracted proximally along the sheath to facilitate capturing tissue within the hood and/or cutting the tissue for resection with the cutting wire.

13. The resection device of claim 12, wherein the device is attached to a distal end of an endoscope.

14. The resection device of claim 9, wherein the cutting wire is an actuation element.

15. The resection device of claim 9, wherein the cutting wire is an electrical path for cautery cutting.

16. The resection device of claim 9, wherein the cutting wire is configured to move between at least one extended state and at least one retracted state, wherein the cutting wire lies along the inner surface of the hood in the extended state and the cutting wire is positioned to bisect the cavity of the hood in the retracted state.

17. The resection device of claim 16, further comprising a groove along the inner surface of the hood wherein the cutting wire can be lodged in the extended state.

18. A method comprising:
placing a snare loop around a target tissue using a tubular shaft having an inner cavity, the snare loop having a single insulated portion and a single conductive portion;
tightening the snare loop around the target tissue by retracting a portion of the snare loop inside the cavity of the shaft; and
activating the snare loop by passing electric current to cut tissue in contact with the conductive portion of the snare loop to make at least a partial perimeter cut around the tissue to be resected.

19. The method of claim 18, further comprising making a plurality of at least partial perimeter cuts around the tissue to be resected, until a full perimeter cut exists around the tissue to be resected.

20. The method of claim 19, further comprising resecting the tissue to be resected by an en bloc technique after making a plurality of perimeter cuts.
